(19) United States
(12) Patent Application Publication
(10) Pub. No.: US 2026/0124461 A1
(43) Pub. Date: May 7, 2026

(54) INTRAORAL PHOTOTHERAPY DEVICE

(71) Applicant: MUREVA PHOTOTHERAPY INC., Strongsville, OH (US)

(72) Inventors: Vedang KOTHARI, San Diego, CA (US); Jordan OJA, Stow, OH (US); Jason LAZZARA, Brooklyn Heights, OH (US); Samuel SHELNUTT, Olmsted Township, OH (US)

(73) Assignee: MUREVA PHOTOTHERAPY INC., Strongsville, OH (US)

(21) Appl. No.: 18/686,283

(22) PCT Filed: Sep. 9, 2022
(86) PCT No.: PCT/US2022/076153

§ 371 Date: Feb. 23, 2024

Related U.S. Application Data
(60) Provisional application No. 63/242,166, filed on Sep. 9, 2021.

(51) Int. Cl. A61N 5/06; A61M 16/00

(52) U.S. Cl. CPC A61N 5/0613; A61M 16/0003; A61M 2205/052; A61N 2005/0606; A61N 2005/063; A61N 2005/0632; A61N 2005/0651; A61N 2005/0659; A61N 2005/0663; A61N 2005/0665

(57) ABSTRACT

An intraoral phototherapy device is provided that improves illumination of a tonsillar region of the oral cavity. Oral tissue illumination is particularly difficult in the back of the throat. The intraoral phototherapy device improves illumination of these tissues by using a breathing tube to improve patient breathing. The breathing tube allows a patient to breathe through their mouth, which opens up the tonsillar region of the oral cavity. Additionally, objects contacting near the back of the throat often induce a gagging reflex. This gagging reflex shortens the time available to perform phototherapy, making it difficult for phototherapy to be delivered to the back of the throat. The intraoral phototherapy device may reduce the gagging reflex by contacting the hard palate of the oral cavity with a dorsal fin, such that the intraoral phototherapy device does not make contact further back in the oral cavity.

### RELATED APPLICATIONS

This application claims the benefit of 63/242,166 filed on Sep. 9, 2021. Which is herein incorporated by reference in its entirety.

### TECHNICAL FIELD

This present disclosure relates generally to phototherapy and more particular to intraoral phototherapy devices.

### BACKGROUND

Phototherapy can be utilized for treating and providing pain relief for various conditions, including a condition called Oral Mucositis (OM). Phototherapy can be delivered in several ways, e.g., directly to the tissue via Low Level Laser Therapy (LLLT) or via a light emitting diode (LED) array that propagates light through the skin into the affected region.

Currently there are two known methods for administering phototherapy for the treatment of various phototherapy treatment conditions of the mouth including, but not limited to Oral Mucositis (OM), low level laser therapy and light emitting diode (LED) arrays. Oral Mucositis is one of the most common and highly significant toxicities of cancer therapy.

Barriers to the acceptance of low-level laser therapy include the cost of laser equipment and the labor intensiveness. Additionally, there are problems with interoperator variability and the need for specialized training. Also, patients receiving this form of treatment are required to hold their mouths open for long periods of time which is uncomfortable and becomes extremely painful as the Mucositis progresses.

LED arrays utilize a plurality of LEDs to irradiate larger areas of tissue externally. The light from these arrays penetrates the skin to stimulate the mucosal membrane. LED arrays have the advantage of irradiating a large surface area, are simpler to implement than spot laser systems, and are more comfortable to the patient. The main disadvantages of using LED arrays for administering phototherapy treatment is that they lack dose control because they must transilluminate cheek tissue and have difficulty reaching all regions of the oral cavity, including the tonsillar and palatal regions which are highly susceptible to OM. Also, variability in tissue thickness between different buccal regions and different patients makes it impossible to accurately monitor and control the dose of light administered to the mucosa.

### SUMMARY

Many patients experience painful lesions at the back of the throat and tonsillar region of the oral cavity caused by oral mucositis. However, it is difficult to treat these effected areas because it is difficult to reach these areas with phototherapeutic light.

The present disclosure provides an intraoral phototherapy device for illuminating a tonsillar region of the oral cavity by using a breathing tube to improve patient breathing and, consequently, open up the tonsillar region for improve illumination of tissues located at the back of the throat.

The present disclosure also provides an intraoral phototherapy device for illuminating a tonsillar region of the oral cavity by using a dorsal projection to interact with the roof of the oral cavity, reducing gagging and opening up the tonsillar region for improve illumination of tissues located at the back of the throat.

The present disclosure also provides a trifurcated sleeve for receiving an intraoral phototherapy device for providing a barrier between the intraoral phototherapy device and the oral cavity, while maintaining the sleeve near the intraoral phototherapy device to improve patient breathing and, consequently, open up the tonsillar region for improved illumination of tissues located at the back of the throat.

While several features are described herein with respect to embodiments of the invention; features described with respect to a given embodiment also may be employed in connection with other embodiments. The following description and the annexed drawings set forth certain illustrative embodiments of the invention. These embodiments are indicative, however, of but a few of the many ways in which the principles of the invention may be employed. Other objects, advantages, and novel features according to aspects of the invention will become apparent from the following detailed description when considered in conjunction with the drawings.

### BRIEF DESCRIPTION OF THE DRAWINGS

The annexed drawings, which are not necessarily to scale, show various aspects of the invention in which similar reference numerals are used to indicate the same or similar parts in the various views.

FIG. 1 is a front perspective view of an embodiment of an intraoral phototherapy system including an intra-oral phototherapy device and an intra-oral phototherapy device protection system having a breathing apparatus and a protective sleeve.

FIG. 2 is a front perspective view of the intra-oral phototherapy device and an exploded view of the breathing apparatus of FIG. 1.

FIG. 3 is a front perspective view of the intra-oral phototherapy device separated from the breathing apparatus of FIGS. 1 and 2.

FIG. 4 is a front perspective view of the breathing apparatus and the protective sleeve of FIG. 1.

FIG. 5 is a front perspective view of an alternative embodiment of the breathing apparatus.

FIG. 6 is a side perspective view of the breathing apparatus of FIG. 5.

FIG. 7 is a rear perspective view of the breathing apparatus of FIGS. 5 and 6.

FIG. 8 is a bottom perspective view of the breathing apparatus of FIGS. 5-7.

FIG. 9 is a front perspective view of a bottom piece of the breathing apparatus of FIGS. 5-8.

FIG. 10 is a bottom perspective view of the bottom piece of FIG. 9.

FIG. 11 is a side perspective view of the bottom piece of FIGS. 9 and 10.

FIG. 12 is a front perspective view of a top piece of the breathing apparatus of FIGS. 5-8.

FIG. 13 is a bottom perspective view of the top piece of FIG. 12.

FIG. 14 is a side perspective view of the top piece of FIGS. 12 and 13.

FIG. 15 is a front perspective view of an embodiment of the intra-oral phototherapy device.

FIG. 16 is a top perspective view of the intra-oral phototherapy device of FIG. 15.

FIG. 17 is a bottom perspective view of the intra-oral phototherapy device of FIGS. 15 and 16.

FIG. 18 is a side perspective view of the intra-oral phototherapy device of FIGS. 15-17.

FIG. 19 is a front perspective view of the intra-oral phototherapy device of FIG. and the breathing apparatus of FIG. 5.

FIG. 20 is a side perspective view of the intra-oral phototherapy device of FIG. 15 and the breathing apparatus of FIG. 5.

FIG. 21 is a top view of the intra-oral phototherapy device of FIG. 15 and the breathing apparatus of FIG. 5.

FIG. 22 is a top front view of the intra-oral phototherapy device of FIG. 15 and the breathing apparatus of FIG. 5.

FIG. 23 is a bottom front view of the intra-oral phototherapy device of FIG. 15 and the breathing apparatus of FIG. 5.

FIG. 24 is a top view of the protective sleeve being manufactured.

FIG. 25 is a view of an unprocessed sleeve showing a position of cuts and connecting of the cuts to form a protective sleeve

FIG. 26 shows a protective sleeve and a breathing apparatus including breathing tubes.

FIG. 27 shows the breathing apparatus of FIG. 26 being attached to the protective sleeve.

FIG. 28 is a top view of the protective sleeve and breathing apparatus of FIGS. 26 and 27.

FIG. 29 shows the intra-oral phototherapy device being inserted into the protective sleeve of FIG. 28.

FIG. 30 shows an alternative embodiment of a breathing apparatus attached to the protective sleeve.

FIG. 31 shows an embodiment of a breathing apparatus attached to the intra-oral phototherapy device.

FIG. 32 is a side perspective view of a light guide of the intra-oral phototherapy device.

FIG. 33 is a side view of the light guide of FIG. 32.

FIG. 34 is a top view of the light guide of FIG. 32.

FIG. 35 is a bottom view of the light guide of FIG. 32.

FIG. 36 is a top view of the light guide of FIG. 32 inserted into the oral cavity.

FIG. 37 is a side view of the light guide of FIG. 32 inserted into the oral cavity.

The present invention is described below in detail with reference to the drawings. In the drawings, each element with a reference number is similar to other elements with the same reference number independent of any letter designation following the reference number. In the text, a reference number with a specific letter designation following the reference number refers to the specific element with the number and letter designation and a reference number without a specific letter designation refers to all elements with the same reference number independent of any letter designation following the reference number in the drawings.

### DETAILED DESCRIPTION

According to an exemplary embodiment, an intraoral phototherapy device is provided that improves illumination of a tonsillar region of the oral cavity. Oral tissue illumination is particularly difficult in the back of the throat. The intraoral phototherapy device improves illumination of these tissues by using a breathing tube to improve patient breathing. The breathing tube allows a patient to breathe through their mouth, which opens up the tonsillar region of the oral cavity. This opening of the tonsillar region allows light emitted from the intra-oral phototherapy device to illuminate tissues located at the back of the throat.

Additionally, objects contacting near the back of the throat often induce a gagging reflex. This gagging reflex shortens the time available to perform phototherapy, making it difficult for phototherapy to be delivered to the back of the throat. The intraoral phototherapy device may reduce the gagging reflex by contacting the hard palate of the oral cavity with a dorsal fin, such that the intraoral phototherapy device does not make contact further back in the oral cavity. That is, the intraoral phototherapy device may include a dorsal fin configured to contact the oral cavity further from the throat, such that portions of the intraoral phototherapy device nearer the throat do not contact the oral cavity and induce a gagging reflex.

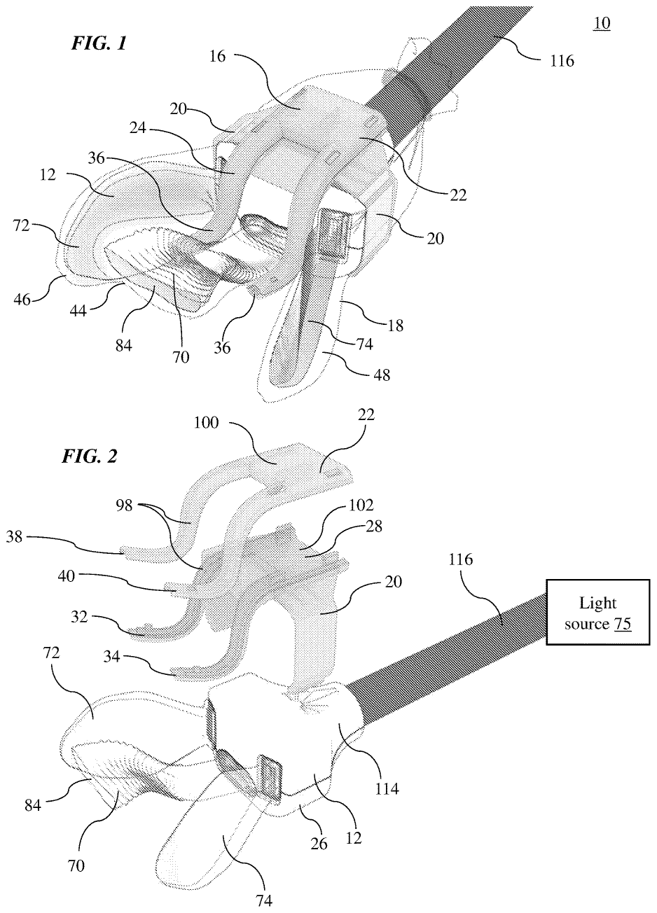

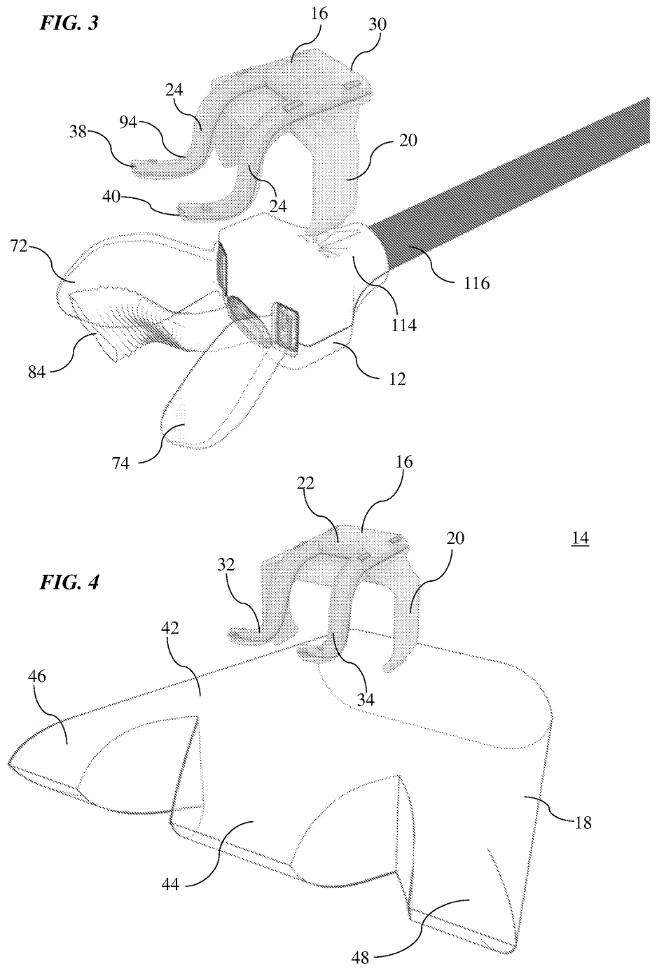

Turning to FIGS. 1-4, an exemplary embodiment of a phototherapy system 10 for illuminating a tonsillar region of an oral cavity is shown. The phototherapy system 10 includes an intra-oral phototherapy device 12 and an intra-oral phototherapy device protection system 14. The protection system 14 includes a breathing apparatus 16 and a sleeve 18 (also referred to as a protective sleeve).

The breathing apparatus 16 improves illumination of the tonsillar region of the oral cavity of a patient during phototherapy by improving patient breathing while receiving the phototherapy with the intra-oral phototherapy device 12. Without the breathing apparatus 16, the patient may need to breath through their nose, which constricts the back of the throat, making it difficult to illuminate the tonsillar region. Conversely, by using the breathing apparatus 16, the patient may breathe through their mouth, opening the back of the throat and improving illumination of the tonsillar region.

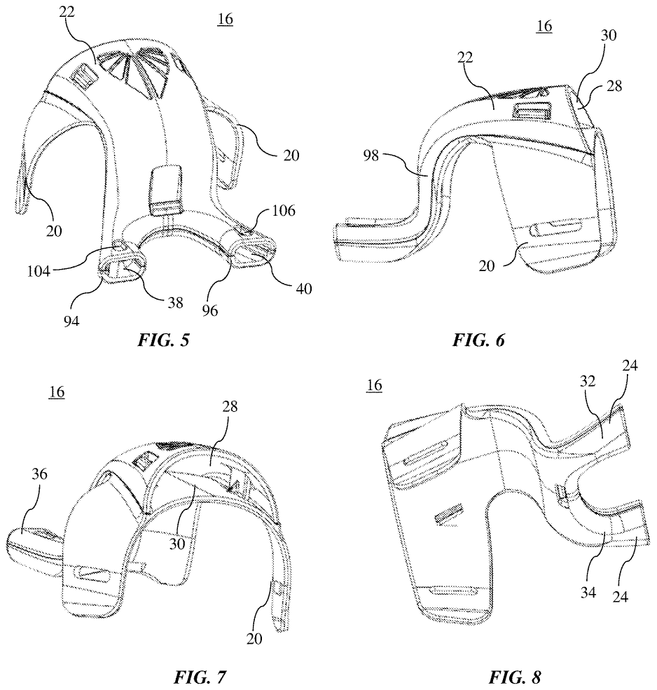

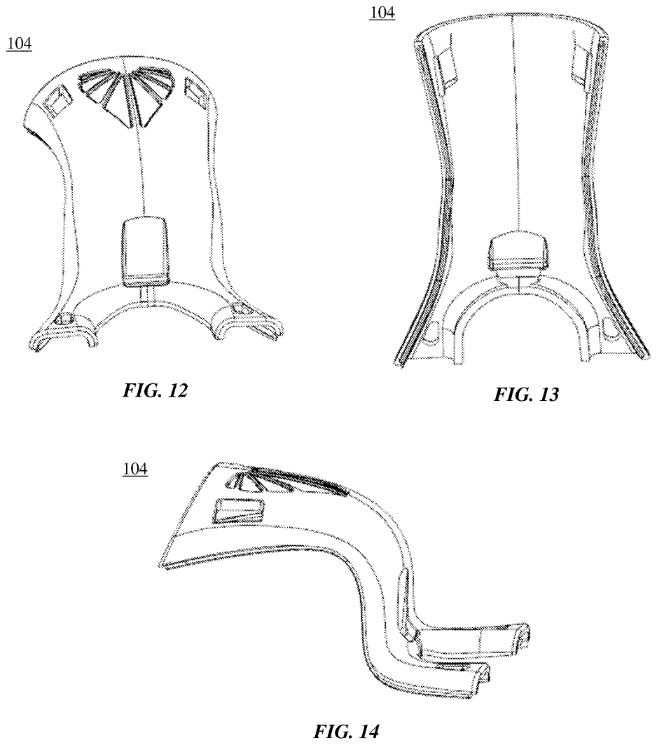

As shown in FIGS. 5-14, the breathing apparatus 16 includes a fixation structure 20, a main body 22, and a bifurcated protrusion 24. The fixation structure 20 mechanically engages with a mounting structure 26 of the intra-oral phototherapy device 12, such that a position of the breathing apparatus 16 is maintained relative to the intra-oral phototherapy device 12. The main body 22 has a central lumen 28 and a distal opening 30 to the central lumen 28. The bifurcated protrusion 24 includes a first lateral lumen 32, a second lateral lumen 34, and a proximal opening 36. The proximal opening includes a first proximal opening 38 to the first lateral lumen 32 and a second proximal opening 40 to the second lateral lumen 34. The first lateral lumen 32 and the second lateral lumen 34 are fluidly coupled to the central lumen 28.

The proximal opening 36 is configured (e.g., shaped) to be received within the oral cavity when the intra-oral phototherapy device 12 is located within the oral cavity and the fixation structure 20 is mechanically engaged with the intra-oral phototherapy device 12. Similarly, the distal opening 30 is configured (e.g., shaped) to be located outside of the oral cavity when the intra-oral phototherapy device is located within the oral cavity and the fixation structure is mechanically engaged with the intra-oral phototherapy device, such that the oral cavity is fluidly coupled with an external environment via the central lumen, the first lateral lumen, and the second lateral lumen. That is, because the proximal opening 36 and distal opening 30 are fluidly connected and because the proximal opening 36 is located within the oral cavity and the distal opening 30 is located outside the oral cavity, the breathing apparatus 16 provides a passage (i.e., via the central lumen 28) for the patient to breath through their mouth.

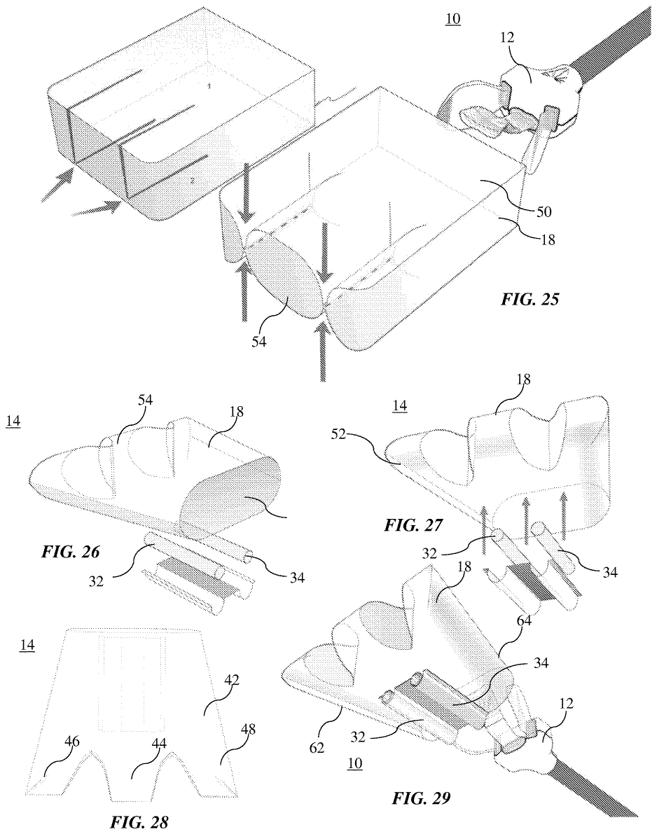

Turning to FIGS. 24 and 25, the protection system 14 includes the sleeve 18. Making the interior volume of the sleeve 18 more form fitting to the intra-oral phototherapy device 12 makes it more difficult to insert the intra-oral phototherapy device 12 into the interior of the sleeve 18. However, making the interior volume larger to improve insertion of the intra-oral phototherapy device 12 into the sleeve 18 also makes it more difficult for the patient to breathe through their mouth with the sleeved intra-oral phototherapy device 12 inside their mouth. By making multiple connected volumes inside the sleeve 18 (e.g., one for each projection of the light guide), it is possible to reduce the bagginess of the sleeve 18 and to improve the patient's ability to breathe through their mouth.

The sleeve 18 is formed from an optically transparent sheet material and has a trifurcated interior volume 42 including a central volume 44, a first lateral volume 46, and a second lateral volume 48. The trifurcated interior volume 42 is formed by folding over the sheet material to form a top sheet 50 and a bottom sheet 52 connected by a closed distal edge 54. The top sheet 50 and the bottom sheet 52 overlap to form an open first lateral edge 56, an open second lateral edge 58, and an open proximal edge 60. The top sheet 50 and the bottom sheet 52 are connected along the first lateral edge 56 to form a closed first lateral edge 62. Similarly, the top sheet 50 and the bottom sheet 52 are connected along the second lateral edge 58 to form a closed second lateral edge 64.

Sheet material is removed from along the closed distal edge 54 to form the central volume 44, the first lateral volume 46, and the second lateral volume 48. That is, a first area 66 of material is removed between the central volume 44 and the first lateral volume 46 and connecting the top sheet 50 and the bottom sheet 52 along a border of the first area 66. Similarly, a second area 68 of material is removed between the central volume 44 and the second lateral volume 48 and connecting the top sheet 50 and the bottom sheet 52 along a border of the second area 68.

The connecting of the top sheet 50 and the bottom sheet 52 forms a seam along the first lateral edge 62 and the second lateral edge 64. This seam may distort light emitted by the intra-oral phototherapy device 12. Conversely, the folding over of the sheet material to form the top sheet 50 and the bottom sheet 52 connected by the closed distal edge 54 does not form a seam along the closed distal edge 54, such that light transmitted through the closed distal edge is not interfered with by a seam. The top sheet 50 and the bottom sheet 52 may be connected using any suitable method (such as heat welding, adhesives, etc.).

The sleeve 18 may be formed in any suitable manner, such that the sleeve 18 does not include a seam along the distal edge 54. For example, the sleeve 18 may be a dip mold or vacuum formed (e.g., similar to a latex glove).

As shown in FIG. 1, the central volume 44 is shaped to receive a light guide 70 of the intra-oral phototherapy device 12. Similarly, the first lateral volume 46 is shaped to receive a first lateral wing 72 and the second lateral volume 48 is shaped to receive a second lateral wing 74 of the intra-oral phototherapy device 12. The fixation structure 20 of the breathing apparatus 16 maintains a position of the sleeve 18 relative to the intra-oral phototherapy device 12 when the fixation structure 20 is mechanically engaged with the intra-oral phototherapy device 12 with the sleeve 18 located between the breathing apparatus 16 and the intra-oral phototherapy device 12. That is, the breathing apparatus 16 may be used to fix the sleeve 18 in position relative to the intra-oral phototherapy device 12.

The sleeve 18 may be made of any suitable material. For example, the sleeve 18 may be disposable and made of a biocompatible plastic sufficient to act as a microbial barrier between the oral cavity and the light guide 70.

As shown in FIGS. 26-31, the breathing apparatus 16 may take different forms. For example, the breathing apparatus 16 may lack a main body and instead include at least one breathing tube 122. In the depicted embodiment, the breathing apparatus 16 includes two breathing tubes 122a, 122b each having a proximal opening that is fluidly connected with a distal opening. The breathing tube(s) 122 may not be fluidly coupled to one another (e.g., the breathing tube(s) 122 may be physically separated).

The breathing tube(s) 122 may be mechanically attached to the sleeve 18 or may be free floating. The breathing tube(s) 122 may also be bendable to enable repositioning of the distal opening(s) (located outside of the oral cavity) relative to the proximal opening(s) (located inside the oral cavity). Alternatively, the breathing tube(s) 122 may have a fixed shape.

The breathing tube(s) 122 may be fixed to the sleeve 18 using tape, by heat welding a piece of the sleeve 18 material to the sleeve 18, or by using any suitable method. For example, the breathing apparatus 16 may be integrated into the sleeve 18 such that the breathing apparatus 16 is mechanically fixed to the sleeve 18. The sleeve 18 may also be sterilized.

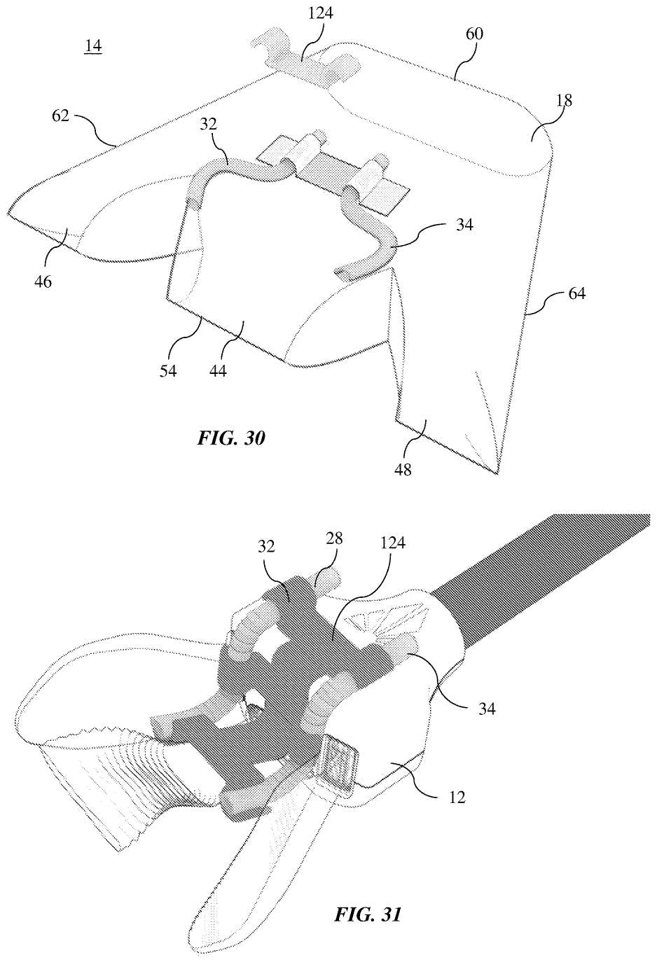

In the embodiments shown in FIGS. 30 and 31, an alternative embodiment of the breathing tubes is shown. In both embodiments, the breathing tubes 122 are bent and attached via a clip 124 to the surface of the sleeve. As shown, the breathing tube(s) 122 may be placed in different positions and/or orientations (e.g., vertically or horizontally) relative to the intraoral phototherapy device 12.

In one embodiment, the breathing apparatus 16 may include protrusion(s) that engage with depression(s) in the intra-oral phototherapy device 12 to maintain a position of the breathing apparatus 16 relative to the intraoral phototherapy device 12.

The breathing apparatus 16 may be configured not to interfere with patient breathing. In one embodiment, the proximal opening(s) of the breathing apparatus 16 may have a cross-sectional area allowing for a tidal volume (Vt) greater than 300 ml. Tidal volume is defined as the amount of air that moves in and out of the lungs with each respiratory cycle (inhalation/exhalation). The breathing apparatus 16 may be configured to allow at least 300 ml of air to be passed through the breathing apparatus 16 without increasing the work of breathing for the patient during a five (5) minute therapy.

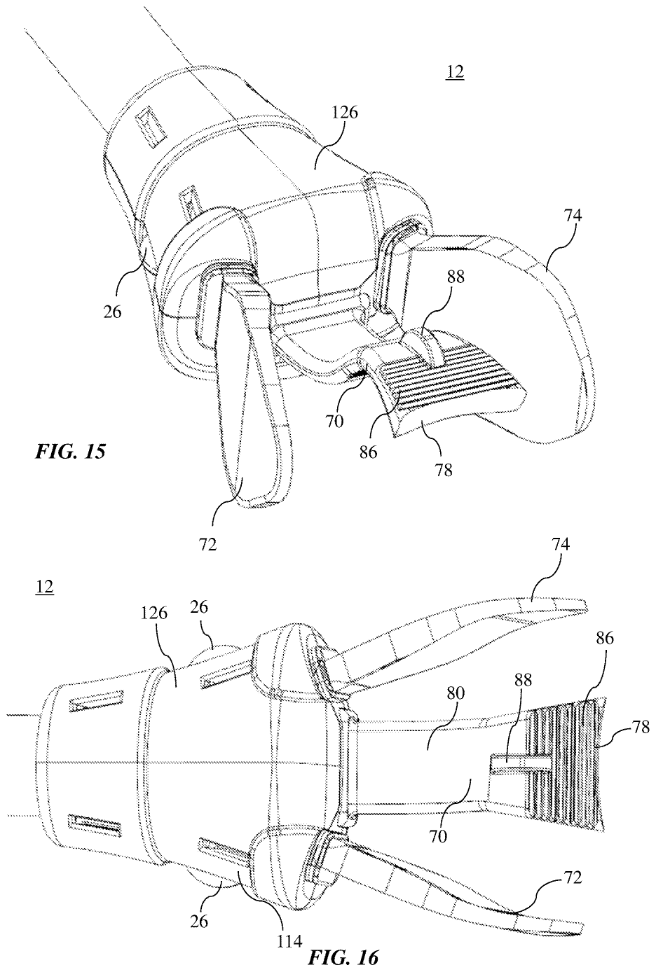

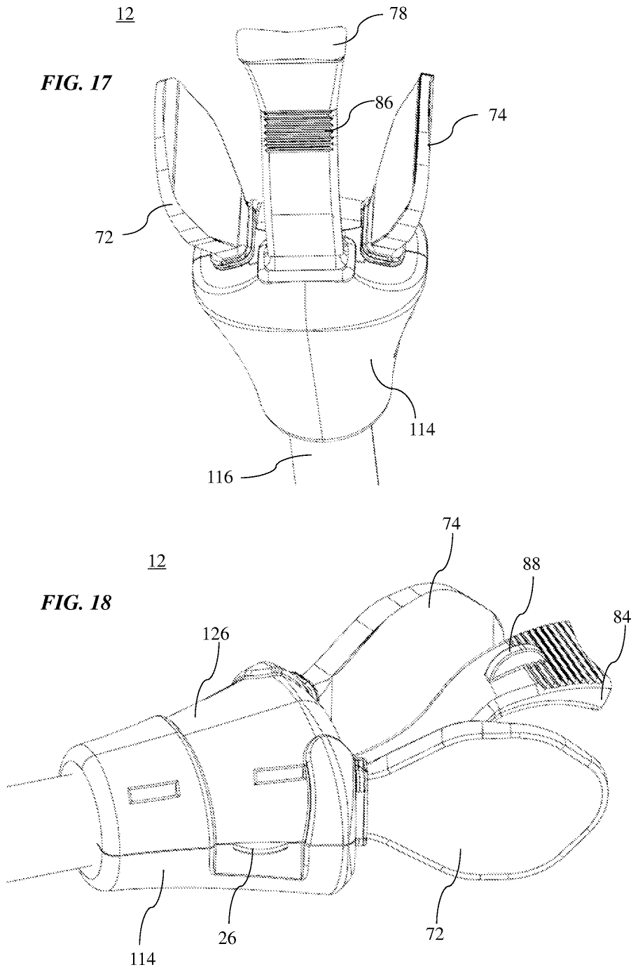

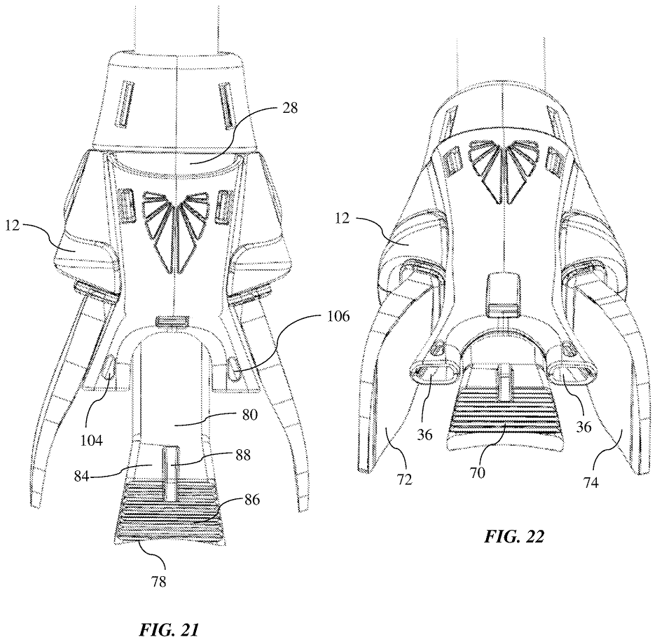

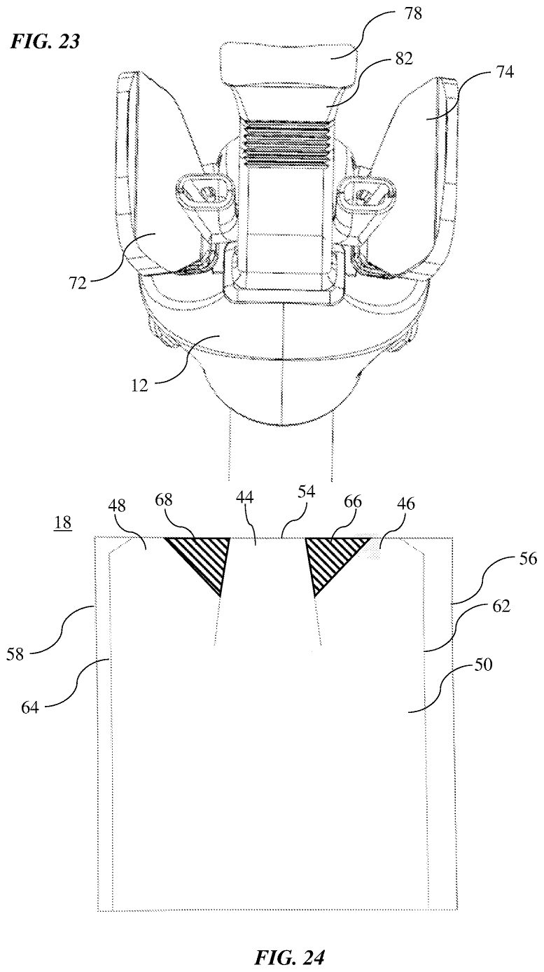

An exemplary embodiment of the intraoral phototherapy device 10 for illuminating targeted regions of the oral cavity with light emitted by a light source 75 is shown in FIGS. 15-23. The intra-oral phototherapy device 10 includes a light guide 70 having a proximal end 76, a distal end 78, a dorsal surface 80, a ventral surface 82, and a main body 84 extending between the proximal end 76, the distal end 78, the dorsal surface 80, and the ventral surface 82.

The light guide 70 receives light from the light source 75 at the proximal end 76 and propagates the received light from the proximal end 76 to the distal 78 end via the main body 84. The main body 84 includes light extracting features 86 that cause light to be emitted from the dorsal surface 80 and the ventral surface 82. The light guide 70 also projects light from the distal end 78. The dorsal surface 80 may have a convex shape and the ventral surface 82 may have a concave shape, such that the main body 84 conforms to contours of the oral cavity when inserted therein to direct light to targeted regions of the oral cavity.

The light guide 70 may also include a dorsal projection 88 extending from the dorsal surface 80 that pushes against a roof of the oral cavity when the intra-oral phototherapy device 12 is inserted into the oral cavity. By interacting with the roof of the oral cavity, the dorsal projection 80 maintains space between the roof of the mouth and the dorsal surface 80 of the intraoral phototherapy device 12 as shown in FIG. 37.

As shown, the dorsal projection 88 is configured to interact with the hard palate and mitigate the leading edge (i.e., the distal end 78) of the light guide 70 from pushing against the upper back of the throat. This improves illumination of the soft palate (also referred to as tonsillar tissues) for phototherapy.

The dorsal projection 88 may take any suitable shape for interacting with the hard palate while mitigating the distal end 78 from pushing against the upper back of the throat. For example, in the depicted figures the dorsal projection 88 has a fin shape. The dorsal projection 88 is not limited to a fin shape but may take any suitable shape (e.g., spherical, elliptical, oblong, etc.).

As described above, the intra-oral phototherapy device 12 may also include lateral wings 72, 74. The lateral wings 72, 74 are optically coupled to the light guide 70 and receive and propagate the light from the light source 75. The first lateral wing 72 (also referred to as a left wing) and the second later wing 74 (also referred to as a right wing) are vertically spaced apart with the light guide 70 is positioned vertically between the first lateral wing 72 and the second lateral wing 74. As shown in FIG. 26, the lateral wings 72, 74 are sized and shaped to be received between buccal tissues and gums of the patient in the oral cavity. That is, the lateral wings 72, 74 include an inner surface 90 facing towards the gums and an outer surface 92 opposite the inner surface 90 facing towards the buccal tissues when inserted into the oral cavity. The lateral wings 72, 74 emit the received light from the inner surface 90 and the outer surface 92.

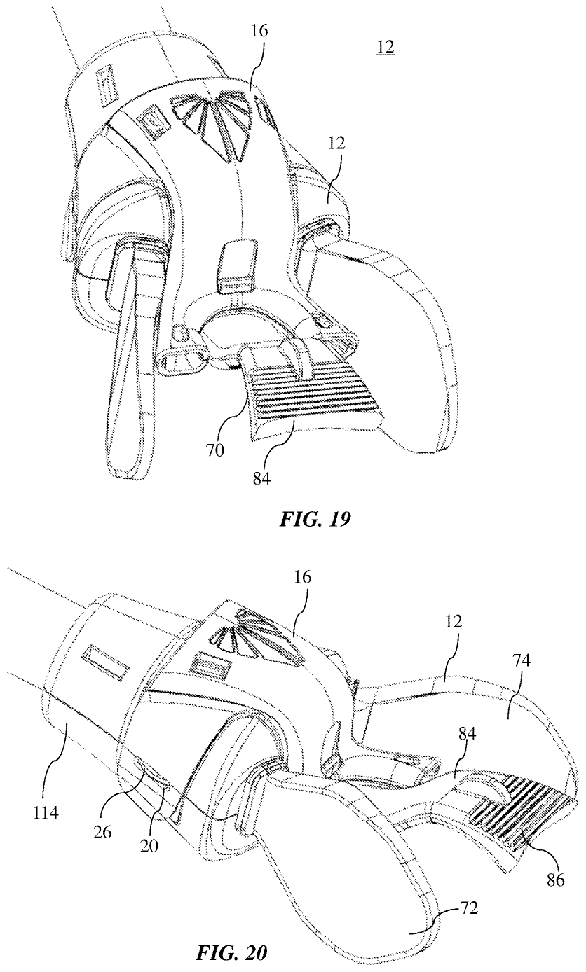

Turning to FIGS. 19-23, the bifurcated protrusion 24 may include a first lateral protrusion 94 enclosing the first lateral lumen 32 and a second lateral protrusion 96 enclosing the second lateral lumen 34. The first lateral protrusion 94 may be separated from the second lateral protrusion 96, such that a central protrusion of the intra-oral phototherapy device is positioned between the first lateral protrusion and the second lateral protrusion when the breathing apparatus is mechanically engaged with the intra-oral phototherapy device.

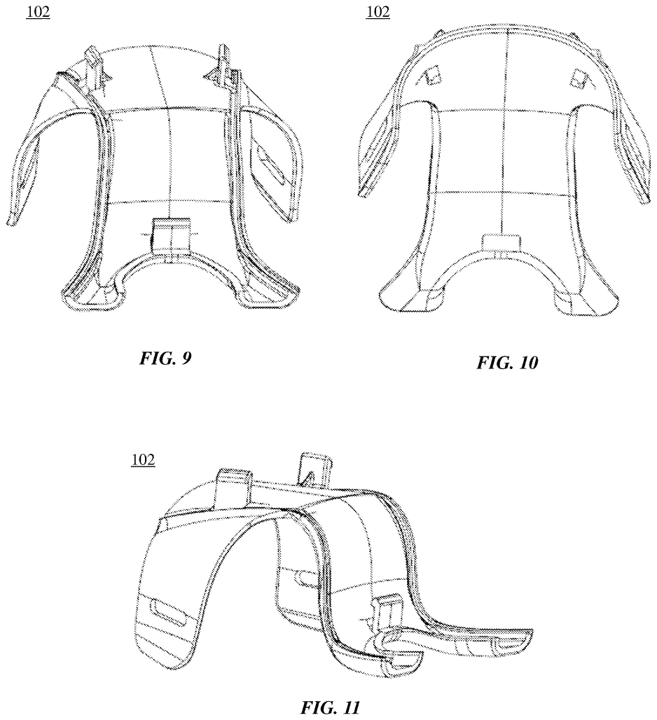

Turning to FIGS. 9-14, the fixation structure 20, the main body 22, and the bifurcated protrusion 24 may be monolithically formed by a housing 98 having a top piece 100 and a bottom piece 102. The bottom piece 102 and the top piece 100 may be shaped to mechanically engage to form the central lumen 28, the first lateral lumen 32, and the second lateral lumen 34. For example, the top piece 100 and bottom piece 102 may snap together. In the depicted example, the bottom piece 102 includes tabs that engage with slots located in the top piece 100.

In one embodiment, the bifurcated protrusion 24 may include a first peripheral opening 104 to the first lateral lumen 32 and a second peripheral opening 106 to the second lateral lumen 34. The first peripheral opening 104 may be located in a different plane than the first proximal opening 38. Similarly, the second peripheral opening 106 may be located in a different plane than the second proximal opening 40. For example, in FIG. 5, the first peripheral opening 104 is located in a plane that is perpendicular to the first proximal opening 38. The peripheral openings 104, 106 may be utilized to improve airflow through breathing apparatus 16.

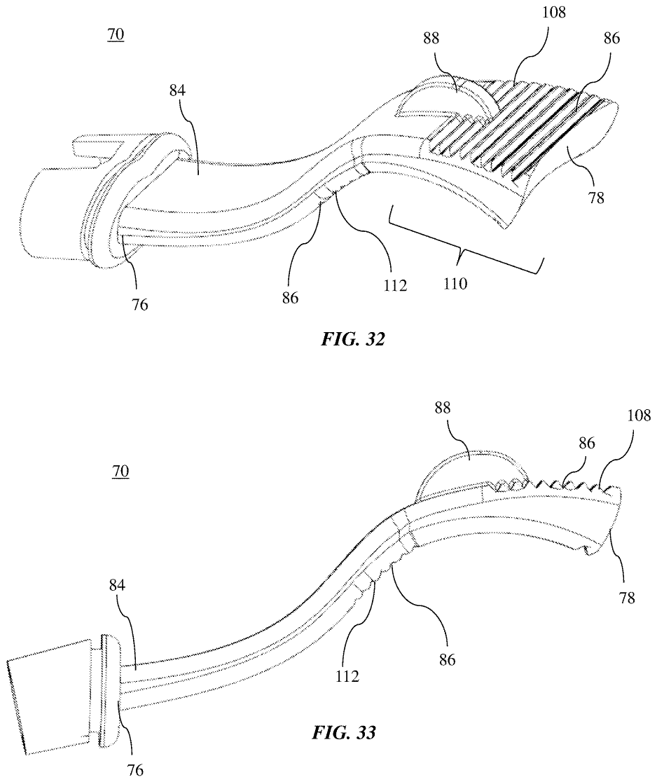

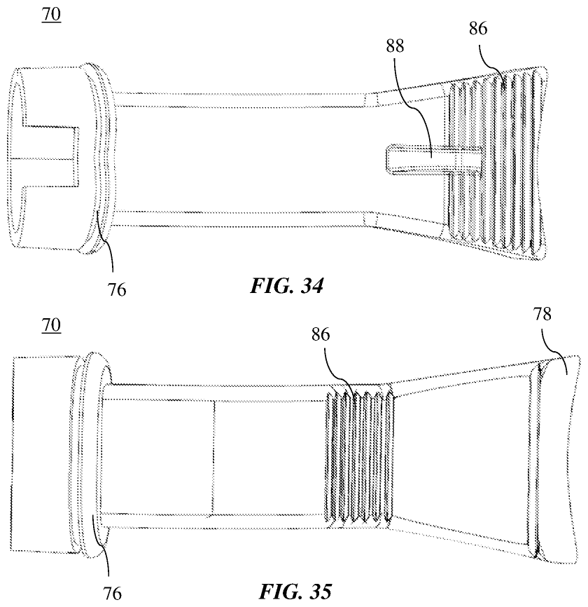

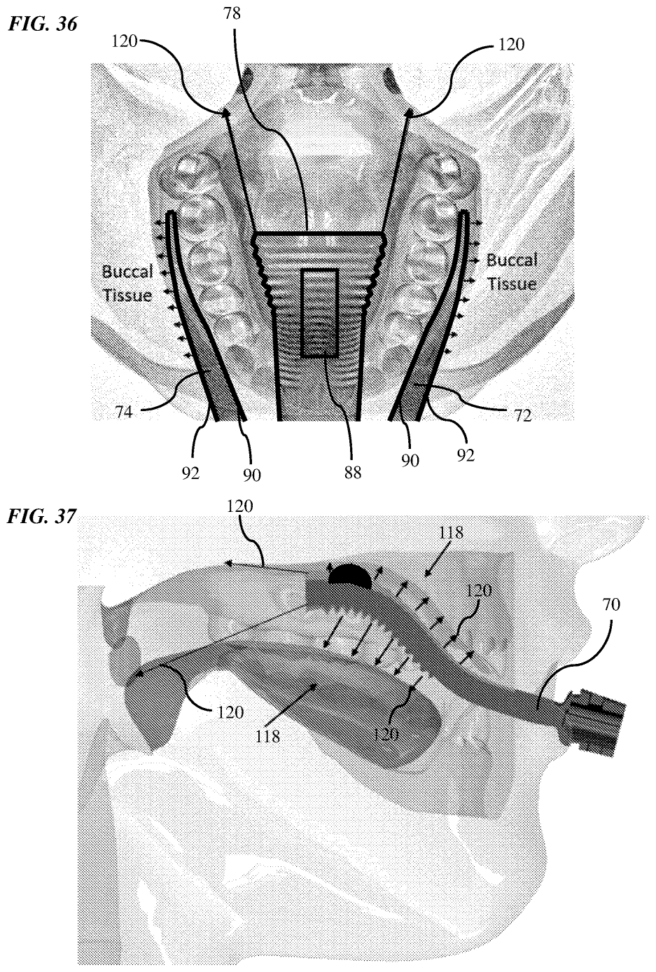

Turning to FIGS. 32-37, the light-extracting features 86 may include dorsal surface features 108 at a distal region 110 of the dorsal surface 80 adjacent the distal end 78. The dorsal surface features 108 may include at least one of projections or depressions in the dorsal surface 80. At least a portion of the dorsal projection 88 may be located in the distal region 110 of the dorsal surface 80. For example, the distal region 110 may comprise one third, one quarter, one fifth, or any suitable portion of the dorsal surface 80 of the light guide 70. The light-extracting features 86 may include ventral surface features on the ventral surface 82 and the ventral surface features 112. The ventral surface features 112 may include at least one of projections or depressions in the ventral surface 82.

The distal end 78 may include projecting optical features configured to direct light from the distal end 78 to the tonsillar tissues. For example, the distal end 78 of the light guide 70 may be contoured to at least partially focus light exiting the distal end 78. That is, the distal end may be configured to emit more focused light from the light guide (e.g., as shown in FIGS. 32-35). The projecting optical features may include any suitable optical structures such as surface shaping lensing, surface aberations, etc.

In a particular embodiment, the ventral surface of the light guide has a curvature that conforms to contours of the dorsal surface of the tongue, such that light emitted from the ventral surface illuminates the dorsal surface of the tongue. The light guide 70 may be contoured to improve patient comfort by following the curve of the roof of the mouth and/or the tongue.

The light-extracting features 86 may be any suitable structure for extracting light from the light guide (e.g., to target a specific light output distribution). For example, the light-extracting 86 features may include at least one of surface aberrations, micro-lenses, reflective spots, partial reflective planes, or diffraction gratings. Alternatively or additionally, a diffuser sheet or a 2-D lensing sheet may be (1) placed on an emission surface of the light guide. In one embodiment, the surface aberrations include at least one of a contour of the surface, surface depositions, or surface etchings.

As shown in FIG. 2, the light source 75 may be external to and not physically supported by the light guide 70 or a casing 114 that the light guide 70 is mechanically attached to. For example, the light source 75 may be a light box supported by an external structure (e.g., table) that is optically connected to the light guide 70 via a light cable 116 (e.g., fiber optics). Alternatively, the light source 75 may be physically supported by the casing 114. For example, the light source 75 may be housed in the casing 114 and powered by a power source electrically connected to the light source 75 (e.g., a battery). Alternatively or additionally, the light source 75 may be mechanically supported by the light guide 70 (e.g., light emitting diodes (LEDs) embedded and/or affixed to the light guide 70). In another example, the light source may be physically mounted to a tab (e.g., a protrusion) on an external surface of the light guide.

In one embodiment, the casing 114 of the intra-oral phototherapy device 12 includes an interface 126 for receiving the main body 22 of the breathing apparatus 16. As shown in FIGS. 15, 16, and 18, the interface 126 may be an indentation or depression shaped to receive a portion of the main body 22.

In one embodiment, the light source is a remote light source that is optically coupled to rearwardly protruding ends of the light guide via a fiber optic cable. In an embodiment, the remote light source includes one or more LEDs or a laser.

The light source 75 may be any suitable structure for emitting light. For example, the light source 75 may include one or more light emitting diodes (LEDs), organic LEDs (OLEDs), microLEDs, laser diodes, mini-LED, quantum dot (QD)-conversion, phosphor conversion, excimer lamps, multi-photon combination, or SLM wavefront manipulation. The light source 75 may emit light (also referred to as electromagnetic radiation) having a wavelength from 600 nm to 1000 nm. For example, the light source 75 may emit light having a wavelength approximately equal to at least one of 630 nm, 660 nm, 670 nm, 810 nm, or 880 nm.

The intra-oral phototherapy device 12 may be configured to illuminated targeted regions 118 of the oral cavity including tissues in addition to the tonsillar tissues. For example, the targeted regions 118 of the oral cavity may include the tonsillar tissues and at least one of the tongue, mandibular and maxillary buccal surfaces of the oral cavity, the floor and roof of the oral cavity, and tonsillar tissues. In one embodiment, the targeted regions 118 of the oral cavity include the tongue, mandibular and maxillary buccal surfaces of the oral cavity, the floor and roof of the oral cavity, and tonsillar tissues.

The light guide 70 may be made of any suitable material that is at least partially transparent to light 120 emitted by the light source 75. For example, the light guide may be made of an optically transparent soft flexible biocompatible polymeric material such as silicone. As an example, the light guide may be made of different formulations of polycarbonate, polymethyl methacrylate, polystyrene, nylon, acrylonitrile butadiene styrene, polyolefin, or other biocompatible thermoplastic elastomer formulations.

The intraoral phototherapy device may be used in several applications, several examples of which include oral mucositis, acute necrotizing ulcerative gingivitis (ANUG), periodontal diseases, trismus, decreasing recovery time from oral surgery, light delivery for orthodontics, and photodynamic light therapy, e.g., to activate a chemical mouthwash.

In one embodiment of the phototherapy system 10, the intra-oral phototherapy device 12 includes a light guide 70 having a proximal end 76, a distal end 78, a dorsal surface 80, a ventral surface 82, and a main body 84 extending between the proximal end 76, the distal end 78, the dorsal surface 80, and the ventral surface 82. The light guide 70 receives light 120 from the light source 75 at the proximal end 76 and propagates the received light 120 from the proximal end 76 to the distal end 78 via the main body 84. The main body 84 includes light extracting features 86 for causing light to be emitted from the dorsal surface 80 and the ventral surface 82. The light guide 70 projects light from the distal end 78. The light guide 70 may take any suitable shape for projecting light into the oral cavity, such as spherical, elliptical, S shaped, etc.

This embodiment of the phototherapy system 10 also includes a breathing apparatus 16 having a fixation structure 20 for mechanically engage with the mounting structure of the intra-oral phototherapy device 12, and a main body 22 having a central lumen 28, a distal opening 30 to the central lumen 28, and a proximal opening 36 to the central lumen 28. For example, in this embodiment, the breathing apparatus 16 may comprise a single breathing tube 122. As an example, the breathing apparatus 16 may be a single breathing located above, below, to the side of the light guide 70. As another example, the light guide 70 may have a central opening and the breathing tube 122 may be formed by or located in this central opening.

All ranges and ratio limits disclosed in the specification and claims may be combined in any manner. Unless specifically stated otherwise, references to “a,” “an,” and/or “the” may include one or more than one, and that reference to an item in the singular may also include the item in the plural.

Although the invention has been shown and described with respect to a certain embodiment or embodiments, equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A breathing apparatus for improving illumination of a tonsillar region of an oral cavity of a patient during phototherapy by improving patient breathing while receiving the phototherapy with an intra-oral phototherapy device, wherein the breathing apparatus includes:
a fixation structure configured to mechanically engage with a mounting structure of the intra-oral phototherapy device, such that a position of the breathing apparatus is maintained relative to the intra-oral phototherapy device;
a main body including a first lateral lumen, a second lateral lumen, a first proximal opening and a first distal opening to the first lateral lumen, and a second proximal opening and a second distal opening to the second lateral lumen, wherein:
the first proximal opening and the second proximal opening are configured to be received within the oral cavity when the intra-oral phototherapy device is located within the oral cavity and the fixation structure is mechanically engaged with the intra-oral phototherapy device; and
the first distal opening and the second distal opening are is configured to be located outside of the oral cavity when the intra-oral phototherapy device is located within the oral cavity and the fixation structure is mechanically engaged with the intra-oral phototherapy device, such that the oral cavity is fluidly coupled with an external environment via the first lateral lumen, and the second lateral lumen.

2. The breathing apparatus of claim 1, wherein:
the first lateral lumen and the second lateral lumen are included in a bifurcated protrusion;
the bifurcated protrusion includes a first lateral protrusion enclosing the first lateral lumen and a second lateral protrusion enclosing the second lateral lumen; and
the first lateral protrusion is separated from the second lateral protrusion, such that a central protrusion of the intra-oral phototherapy device is positioned between the first lateral protrusion and the second lateral protrusion when the breathing apparatus is mechanically engaged with the intra-oral phototherapy device.

3. The breathing apparatus of claim 1, wherein:
the fixation structure, the main body, and the bifurcated protrusion are monolithically formed by a housing having a top piece and a bottom piece; and
the bottom piece and the top piece are shaped to mechanically engage to form the central lumen, the first lateral lumen, and the second lateral lumen.

4. The breathing apparatus of claim 1, wherein:
the bifurcated protrusion further includes a first peripheral opening to the first lateral lumen and a second peripheral opening to the second lateral lumen;
the first peripheral opening is located in a different plane than the first proximal opening; and
the second peripheral opening is located in a different plane than the second proximal opening.

5. An intra-oral phototherapy device protection system for providing a barrier between the received light guide and an oral cavity of a patient, the system comprising:
the breathing apparatus of claim 1;
a sleeve formed from an optically transparent sheet material and having a trifurcated interior volume including a central volume, a first lateral volume, and a second lateral volume, wherein the trifurcated interior volume is formed by:
folding over the sheet material to form a top sheet and a bottom sheet connected by a closed distal edge, wherein the top sheet and the bottom sheet overlap to form an open first lateral edge, an open second lateral edge, and an open proximal edge;
connecting the top sheet and the bottom sheet along the first lateral edge to form a closed first lateral edge;
connecting the top sheet and the bottom sheet along the second lateral edge to form a closed second lateral edge;
removing sheet material from along the closed distal edge to form the central volume, the first lateral volume, and the second lateral volume by:
removing a first area of material between the central volume and the first lateral volume and connecting the top sheet and the bottom sheet along a border of the first area; and
removing a second area of material between the central volume and the second lateral volume and connecting the top sheet and the bottom sheet along a border of the second area;
wherein the central volume is shaped to receive a light guide of the intra-oral phototherapy device;
wherein the first lateral volume is shaped to receive a first lateral wing of the intra-oral phototherapy device; and
wherein the second lateral volume is shaped to receive a second lateral wing of the intra-oral phototherapy device; and
wherein the fixation structure of the breathing apparatus is configured to maintain a position of the sleeve relative to the intra-oral phototherapy device when the fixation structure is mechanically engaged with the intra-oral phototherapy device.

6. The system of claim 5, wherein:
the connecting of the top sheet and the bottom sheet along the first lateral edge to form the first lateral edge forms a seam along the first lateral edge;
the connecting of the top sheet and the bottom sheet along the second lateral edge to form the second lateral edge forms a seam along the second lateral edge;
the folding over of the sheet material to form the top sheet and the bottom sheet connected by the closed distal edge does not form a seam along the closed distal edge, such that light transmitted through the closed distal edge is not interfered with by a seam.

7. The system of claim 5, wherein the breathing apparatus is integrated into the sleeve such that the breathing apparatus is mechanically fixed to the sleeve.

8. An intraoral phototherapy device for illuminating targeted regions of an oral cavity of a patient with light emitted by a light source, the device comprising:
a light guide having a proximal end, a distal end, a dorsal surface, a ventral surface, and a main body extending between the proximal end, the distal end, the dorsal surface, and the ventral surface, wherein:
the light guide is configured to receive light from the light source at the proximal end and to propagate the received light from the proximal end to the distal end via the main body;
the main body includes light extracting features configured to cause light to be emitted from the dorsal surface and the ventral surface;
the light guide is configured to project light from the distal end;
the dorsal surface has a convex shape and the ventral surface has a concave shape, such that the main body conforms to contours of the oral cavity when inserted therein to direct light to targeted regions of the oral cavity; and
a dorsal projection extending from the dorsal surface and configured to push against a roof of the oral cavity when the intra-oral phototherapy device is inserted into the oral cavity.

9. The device of claim 8, wherein:
the light-extracting features includes dorsal surface features at a distal region of the dorsal surface adjacent the distal end;
the dorsal surface features include at least one of projections or depressions in the dorsal surface; and
at least a portion of the dorsal projection is located in the distal region of the dorsal surface.

10. The device of claim 8, wherein the light-extracting features include ventral surface features on the ventral surface and the ventral surface features include at least one of projections or depressions in the ventral surface.

11. The device of claim 8, wherein the light source is external to and not physically supported by the light guide.

12. The device of claim 8, wherein the targeted regions of the oral cavity include at least one of the tongue, mandibular and maxillary buccal surfaces of the oral cavity, the floor and roof of the oral cavity, and tonsillar tissues.

13. The device of claim 8, wherein the targeted regions of the oral cavity include the tongue, mandibular and maxillary buccal surfaces of the oral cavity, the floor and roof of the oral cavity, and tonsillar tissues.

14. The device of claim 8, wherein the light guide is made of a material that is at least partially transparent to light emitted by the light source.

15. The device of claim 8, further comprising:
lateral wings optically coupled to the light guide and configured to receive and propagate the light from the light source, wherein:
the lateral wings include a left wing and a right wing vertically spaced from the left wing; and
the light guide is positioned vertically between the left wing and the right wing;
the lateral wings are sized and shaped to be received between buccal tissues and gums of the patient; and
the lateral wings include an inner surface facing towards the gums and an outer surface opposite the inner surface facing towards the buccal tissues when inserted into the oral cavity, wherein the lateral wings are configured to emit the received light from the inner surface and the outer surface.

16. A phototherapy system for illumination a tonsillar region of an oral cavity of a patient by improving patient breathing while receiving the phototherapy, wherein the system comprises:
the intra-oral phototherapy device of claim 8, further comprising a mounting structure; and
a breathing apparatus including:
a fixation structure configured to mechanically engage with the mounting structure of the intra-oral phototherapy device, such that a position of the breathing apparatus is maintained relative to the intra-oral phototherapy device;
a main body including a first lateral lumen, a second lateral lumen, a first proximal opening and a first distal opening to the first lateral lumen, a second proximal opening and a second distal opening to the second lateral lumen, wherein:
the first lateral lumen and the second lateral lumen are shaped such that the light guide is positioned vertically between the first lateral lumen and the second lateral lumen when the fixation structure is mechanically engaged with the intra-oral phototherapy device;
the first proximal opening and the second proximal opening are is configured to be received within the oral cavity when the intra-oral phototherapy device is located within the oral cavity and the fixation structure is mechanically engaged with the intra-oral phototherapy device; and
the first distal opening and the second distal opening are is configured to be located outside of the oral cavity when the intra-oral phototherapy device is located within the oral cavity and the fixation structure is mechanically engaged with the intra-oral phototherapy device, such that the oral cavity is fluidly coupled with an external environment via the first lateral lumen, and the second lateral lumen.

17. The system of claim 16, further comprising:
a sleeve formed from an optically transparent sheet material and having a trifurcated interior volume including a central volume, a first lateral volume, and a second lateral volume, wherein the trifurcated interior volume is formed by:
folding over the sheet material to form a top sheet and a bottom sheet connected by a closed distal edge, wherein the top sheet and the bottom sheet overlap to form an open first lateral edge, an open second lateral edge, and an open proximal edge;
connecting the top sheet and the bottom sheet along the first lateral edge to form a closed first lateral edge;
connecting the top sheet and the bottom sheet along the second lateral edge to form a closed second lateral edge;
removing sheet material from along the closed distal edge to form the central volume, the first lateral volume, and the second lateral volume by:
removing a first area of material between the central volume and the first lateral volume and connecting the top sheet and the bottom sheet along a border of the first area; and
removing a second area of material between the central volume and the second lateral volume and connecting the top sheet and the bottom sheet along a border of the second area;
wherein the central volume is shaped to receive the light guide of the intra-oral phototherapy device;
wherein the first lateral volume is shaped to receive a first lateral wing of the intra-oral phototherapy device; and
wherein the second lateral volume is shaped to receive a second lateral wing of the intra-oral phototherapy device; and
wherein the fixation structure of the breathing apparatus is configured to maintain a position of the sleeve relative to the intra-oral phototherapy device when the fixation structure is mechanically engaged with the intra-oral phototherapy device.

18. The system of claim 17, wherein:
the connecting of the top sheet and the bottom sheet along the first lateral edge to form the first lateral edge forms a seam along the first lateral edge;
the connecting of the top sheet and the bottom sheet along the second lateral edge to form the second lateral edge forms a seam along the second lateral edge;
the folding over of the sheet material to form the top sheet and the bottom sheet connected by the closed distal edge does not form a seam along the closed distal edge, such that light transmitted through the closed distal edge is not interfered with by a seam.

19. A phototherapy system for illuminating a tonsillar region of an oral cavity of a patient by improving patient breathing while receiving the phototherapy, wherein the system comprises:
an intraoral phototherapy device for illuminating targeted regions of an oral cavity of a patient with light emitted by a light source, the device comprising:
a light guide having a proximal end, a distal end, a dorsal surface, a ventral surface, and a main body extending between the proximal end, the distal end, the dorsal surface, and the ventral surface, wherein:
the light guide is configured to receive light from the light source at the proximal end and to propagate the received light from the proximal end to the distal end via the main body;
the main body includes light extracting features configured to cause light to be emitted from the dorsal surface and the ventral surface;
the light guide is configured to project light from the distal end;
a breathing apparatus including:
a fixation structure configured to mechanically engage with the mounting structure of the intra-oral phototherapy device, such that a position of the breathing apparatus is maintained relative to the intra-oral phototherapy device; and
a main body having a central lumen, a distal opening to the central lumen, and a proximal opening to the central lumen, wherein:
the proximal opening is configured to be received within the oral cavity when the intra-oral phototherapy device is located within the oral cavity and the fixation structure is mechanically engaged with the intra-oral phototherapy device; and
the distal opening is configured to be located outside of the oral cavity when the intra-oral phototherapy device is located within the oral cavity and the fixation structure is mechanically engaged with the intra-oral phototherapy device, such that the oral cavity is fluidly coupled with an external environment via the central lumen, the first lateral lumen, and the second lateral lumen.

20. The system of claim 19, wherein:
the main body further includes a bifurcated protrusion;
the bifurcated protrusion includes the proximal opening, a first lateral lumen, and a second lateral lumen;
the proximal opening comprises a first proximal opening to the first lateral lumen and a second proximal opening to the second lateral lumen;
the first lateral lumen and the second lateral lumen are fluidly coupled to the central lumen; and
the first lateral lumen and the second lateral lumen are shaped such that the light guide is positioned vertically between the first lateral lumen and the second lateral lumen when the fixation structure is mechanically engaged with the intra-oral phototherapy device.

21. The system of claim 19, wherein the dorsal surface of the light guide has a convex shape and the ventral surface of the light guide has a concave shape, such that the main body conforms to contours of the oral cavity when inserted therein to direct light to targeted regions of the oral cavity.

22. The breathing apparatus of claim 1, wherein:
the main body has a central lumen and a distal opening to the central lumen;
the first lateral lumen and the second lateral lumen are fluidly coupled to the central lumen; and
the distal opening of the central lumen is formed by the first distal opening of the first lateral lumen and the second distal opening of the second lateral lumen.

23. The phototherapy system of claim 17, wherein:
the main body has a central lumen and a distal opening to the central lumen;
the first lateral lumen and the second lateral lumen are fluidly coupled to the central lumen; and
the distal opening of the central lumen is formed by the first distal opening of the first lateral lumen and the second distal opening of the second lateral lumen.
